(19) United States
(12) Patent Application Publication
(10) Pub. No.: US 2020/0058393 A1
(43) Pub. Date: Feb. 20, 2020

(54) RESOURCE ALLOCATION METHOD, APPARATUS, SYSTEM, ELECTRONIC DEVICE AND STORAGE MEDIUM

(71) Applicants: BEIJING BOE DISPLAY TECHNOLOGY CO., LTD., Beijing (CN); BOE TECHNOLOGY GROUP CO., LTD., Beijing (CN)

(72) Inventors: Ruirui WANG, Beijing (CN); Xingliang LI, Beijing (CN); Yingqiang GAO, Beijing (CN); Huabin CHEN, Beijing (CN); Yongzhi SONG, Beijing (CN)

(21) Appl. No.: 16/448,614

(22) Filed: Jun. 21, 2019

(30) Foreign Application Priority Data
Aug. 15, 2018 (CN) 201810929760.9

(51) Int. Cl. G16H 40/20; G16H 80/00; G06Q 10/10; G06Q 10/02; G16H 10/60

(52) U.S. Cl. CPC G16H 40/20; G16H 80/00; G16H 10/60; G06Q 10/02; G06Q 10/1095

(57) ABSTRACT

There is provided in embodiments of the present disclosure a resource allocation method, apparatus, system, an electronic device and storage medium. The resource allocation method includes: receiving a resource allocation request of a first terminal and determining a type of the resource allocation request; feeding back an allocation result of an allocation of a first resource to the first terminal and a second terminal respectively, according to the type of the resource allocation request; acquiring feedback information about the allocation result from at least one of the first terminal and the second terminal; feeding back to the first terminal information about whether to modify the allocated first resource to a second resource, according to the feedback information.

### CROSS-REFERENCE TO RELATED APPLICATION

The present disclosure claims the priority of a Chinese patient application No. 201810929760.9 filed on Aug. 15, 2018. Herein, the content disclosed by the Chinese patient application is incorporated in full by reference as a part of the present disclosure.

### TECHNICAL FIELD

The present disclosure relates to a technical field of health information management, in particular to a resource allocation method, apparatus, system, an electronic device and a storage medium.

### BACKGROUND

At present, in most hospitals, the registration mode is a separate registration platform available for patients as a single hospital. If a patient has a demand to see a doctor, he/she has to visit an on-site registration window of a hospital, or make an online registration on an online registration platform of the hospital. Obviously, the two registration modes correspond to separate and limited medical resources, while effective communication among a plurality of hospitals has not been realized yet. Thus medical resources of the plurality of hospitals cannot be integrated and allocated collectively.

In this situation, if a person suffers from an illness and becomes a patient, he/she would select a relatively higher-level hospital to see a doctor at the first time. For example, most patients prefer to select a first-class hospital at Grade 3 when they get sick. This results in that a relatively higher-level hospital is forced to bear pressure from gathered registrations, and thus the relatively higher-level hospital is overcrowded, which further causes a chain of negative reactions including difficulty in registering at a hospital, difficulty in seeking medical care, and scalpers being rampant. On one hand, some patients having lighter illness occupy resources of the relatively higher-level hospitals. On the other hand, some patients who really need to be treated in high-level hospitals cannot acquire medical resources in time, thereby causing illness delayed. Thus, it can be seen that the prior art has technical problems of non-uniform and unreasonable allocation of medical resources or low overall utilization rate of medical resources.

### SUMMARY

There are provided in the present disclosure a resource allocation method apparatus, an electrode device and a readable storage medium.

According to one aspect of the present disclosure, there is provided a resource allocation method, comprising: receiving a resource allocation request of a first terminal and determining a type of the resource allocation request; feeding back, according to the type of the resource allocation request, an allocation result of allocation of a first resource to the first terminal and a second terminal respectively; acquiring feedback information about the allocation result from at least one of the first terminal and the second terminal; and feeding back to the first terminal, according to the feedback information, information about whether to modify the allocated first resource to a second resource.

In some embodiments, the first terminal is a patient side terminal, the second terminal is a doctor side terminal, and the resource allocation request is a request for seeing a doctor. The resource allocation method comprises: receiving the request for seeing a doctor from the patient side terminal and determining a type of the request for seeing a doctor; feeding back, according to the type of the request for seeing a doctor, the allocation result of an allocation of a designated-level hospital to the doctor side terminal and the patient side terminal respectively; acquiring feedback information about the allocation result from at least one of the doctor side terminal and the patient side terminal; feeding back, according to the feedback information, to the patient side terminal information about whether to refer the patient to a higher-level hospital.

According to another aspect of the present disclosure, there is further provided a resource allocation apparatus, comprising: a resource allocation request type determination module, configured to receive a resource allocation request of a first terminal and determine a type of the resource allocation request; a resource allocation module, configured to feed back, according to the type of the request for seeing a doctor, an allocation result of allocation of a first resource to the first terminal and a second terminal respectively; a feedback processing module, configured to acquire feedback information about the allocation result from at least one of the first terminal and the second terminal and feed back, according to the feedback information, to the first terminal information about whether to modify the allocated first resource to a second resource.

In some embodiments, the first terminal is a patient side terminal, the second terminal is a doctor side terminal, and the resource allocation request is a request for seeing a doctor. The resource allocation apparatus comprises: a seeing-a-doctor request type determination module, configured to receive the request for seeing a doctor from the patient side terminal and determine a type of the request for seeing a doctor; a hospital allocation module, configured to feed back, according to the type of the request for seeing a doctor, the allocation result of allocation of a designated-level hospital to the doctor side terminal and the patient side terminal respectively; a feedback processing module, configured to acquire feedback information about the allocation result from at least one of the doctor side terminal and the patient side terminal, and feed back, according to the feedback information, to the patient side terminal information about whether to refer the patient to a higher-level hospital.

According to another aspect of the present disclosure, there is further provided an electronic device, comprising: a memory and a processor electrically connected with each other; at least one program, stored in the memory and configured to implement the resource allocation method as described above when being executed by the processor.

According to another aspect of the present disclosure, there is further provided a resource allocation system, comprising: a first terminal, a second terminal and a server device, wherein the server device of the system has a communication connection with the first terminal and the second terminal respectively; and the server device comprises the resource allocation apparatus as described above.

According to another aspect of the present disclosure, there is further provided a computer readable storage medium, upon which a computer program is stored. The computer program implements the resource allocation method as described above when being executed by a processor.

Beneficial technical effects brought by the technical solutions provided in the embodiments of the present disclosure are given as follows.

In the embodiments of the present disclosure, the request for seeing a doctor is received at the patient side terminal, and the type of the request for seeing a doctor is determined. According to the type of the request for seeing a doctor, the information about allocation of a designated-level hospital is fed back to the doctor side terminal and the patient side terminal respectively. The technical solutions provided in the present disclosure are capable of arranging requests for seeing a doctor from a great number of patient side terminals at a same starting point, and both seeing a doctor and registering at a hospital need to start from a designated-level of hospital (for example, seeing a doctor at a primary first-level hospital), so that medical resources of respective levels of hospitals are integrated efficiently.

In the embodiments of the present disclosure, it is also necessary to acquire the feedback information about the allocation result from the doctor side terminal and the patient side terminal, and determine whether the patient is referred to a higher-level hospital according to the feedback information of the doctor side terminal and the patient side terminal. Thus it can be seen that the method provided in the present disclosure gives considerations to diagnosis & treatment comments of both the doctor and the patient, so that the doctor has the right to select a patient and give treatment to the patient, or recommend him/her to a higher-level hospital, and the doctor has the right to decentralize the patients when being confronted with the pressure of being overcrowded. At the same time, the patient obtains more benefits and freedoms. The patient has the right to decide whether to see a doctor in a current-level hospital, which not only optimizes the allocation way of the medical resources, but also responds to will of the patients, and satisfies the requirements of the patients as much as possible, so that it is helpful to improve the tense contradictory relationship between the doctors and the patients and reduce occurrence probability of disputes between the doctors and the patients.

Additional aspects and advantages of the present disclosure will be given partially in the following description. These aspects and advantages would become evident according to the following description, or will be known according to the practice of the present disclosure.

### BRIEF DESCRIPTION OF THE DRAWINGS

The above and/or additional aspects and advantages of the present disclosure would become obvious and easy to be understood according to the following description of the embodiments combined with accompanying figures, of which:

FIG. 1A is a schematic diagram of structure of a resource allocation system provided in some embodiments of the present disclosure.

FIG. 1B is a schematic diagram of structure of a medical resource allocation system provided in some embodiments of the present disclosure.

FIG. 2A is a schematic flowchart of a resource allocation method provided in some embodiments of the present disclosure.

FIG. 2B is a schematic flowchart of a medical resource allocation method provided in some embodiments of the present disclosure.

FIG. 3 is a schematic flowchart of an example of a medical resource allocation method provided in some embodiments of the present disclosure.

FIG. 4A is a schematic diagram of structure of a resource allocation apparatus provided in some embodiments of the present disclosure.

FIG. 4B is a schematic diagram of structure of a medical resource allocation apparatus provided in some embodiments of the present disclosure.

FIG. 5 is a schematic diagram of structure of an electrode device provided in some embodiments of the present disclosure.

### DETAILED DESCRIPTION

Inventor(s) of the present disclosure finds that the medical community is confronted with the phenomenon that patients gather together to select the first-class hospital at Grade 3 to see a doctor, thereby resulting in a situation where high-level hospitals are overcrowded, registering at the hospitals and seeing the doctors become difficult, and allocation of medical resources is unequal. Correspondingly, doctors and nurses in a high-level hospital work overload to serve for a great number of patients. As a result, it tends to cause the relationship between the doctors and the patients becomes tense increasingly.

However, among the patients who see a doctor in the high-level hospital, there are a considerable number of patients suffering from non-urgent and normal illnesses, and actually a primary lower-level hospital can satisfy these patients' requirements for seeing a doctor. These patients suffering from non-urgent and normal illnesses are occupying the medical resources of the high-level hospitals, so that many patients who really need to be treated in the high-level hospital cannot register at the high-level hospitals, which causes that the allocation of the medical resources in the present phase are in a non-optimal state where the allocation of the medical resources is non-uniform, unreasonable and has been not fully utilized yet.

In the present phase, although some hospitals can, depending on the condition of a patient, consider to decide to refer the patient to other hospitals on the premise of being agreed by a doctor, most hospitals stipulates that the patient can propose a referral application only if the patient has got the doctor's permission. However, diagnosis and treatment recommendations of the doctor and the hospital play a key role in this referral flow. The will of the patient suffering from an illness makes very little impact on determination of whether to be referred. This neglects the rights and wills of the patient suffering from the illness. As a result, the allocation of resource is short of processing and response to the will of the patient while the allocation of the medical resources is non-uniform or the overall utilization rate of the medical resources is low. Therefore the demand of the patient is not satisfied.

In order to solve the technical problem that the allocation of the medical resources in the present phase is non-uniform and unreasonable, or the overall utilization rate of the medical resources is low, inventor(s) of the present disclosure creatively proposes a method, an apparatus, a system, an electronic device and storage medium for medical resource allocation.

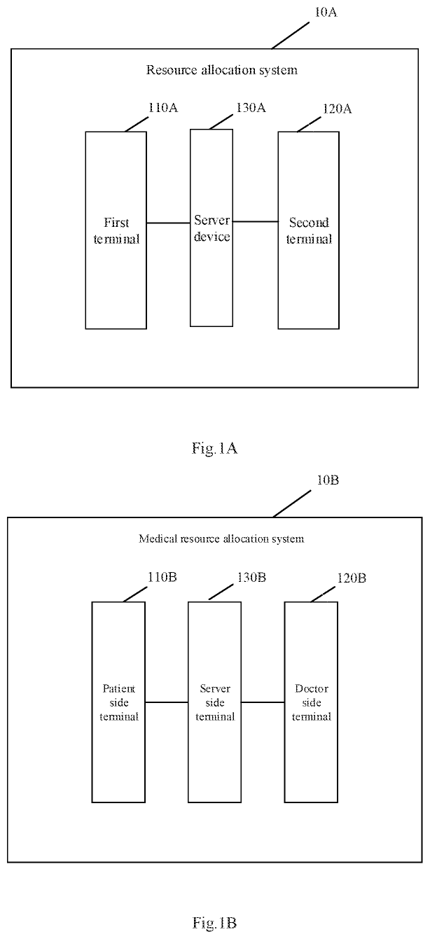

FIG. 1A is schematic diagram of structure of a resource allocation system provided in some embodiments of the present disclosure. Major structure compositions of the resource allocation system 10A will be introduced below by referring to FIG. 1A.

As shown in FIG. 1, the resource allocation system 10A may comprise a first terminal 110A, a second terminal 120A and a server device 130A. The server device 130A of the system 10A has a communication connection with the first terminal 110A and the second terminal 120A respectively, and the server device 130A comprises a resource allocation apparatus (which will be introduced below in details) provided in the embodiments of the present disclosure. For example, the first terminal 110A can be a patient side terminal, which is available for and used by a patient. The second terminal 120A can be a doctor side terminal, which is available for and used by a doctor. By utilizing this system, a resource allocation method provided in the present disclosure which would be described in the following context can be implemented.

FIG. 1B is a schematic diagram of structure of a medical resource allocation system provided in some embodiments of the present disclosure. Major structure compositions of a medical resource allocation system 10B will be introduced below by referring to FIG. 1B.

As shown in FIG. 1B, the medical resource allocation system 10B can comprise: a patient side terminal 110B, a doctor side terminal 120B and a server device 130B. The server device 130B of the system 10B has a communication connection with the patient side terminal 110B and the doctor side terminal 120B respectively, and the server device 130B comprises a medical resource allocation apparatus provided in some embodiments of the present disclosure (which will be introduced below in details). In some embodiments, the patient side terminal is available for and used by the patient, and the doctor side terminal is available for and used by the patient. By utilizing this system, a medical resource allocation method provided according to one aspect of the present disclosure can be implemented.

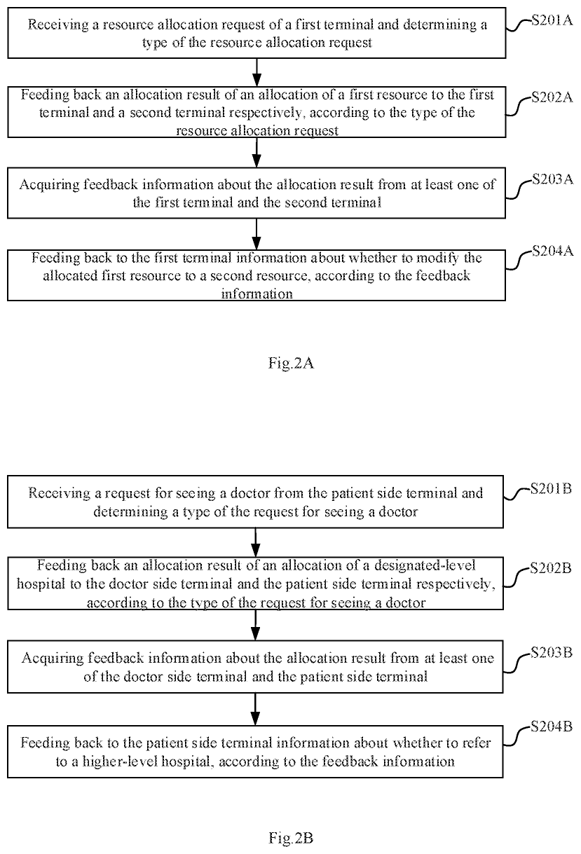

FIG. 2A is a schematic flowchart of a resource allocation method provided in some embodiments of the present disclosure. By referring to FIG. 2A, it is known that in step S201A, a resource allocation request of a first terminal is received, and a type of the resource allocation request is determined. In step S202A, an allocation result of an allocation of a first resource is fed back to the first terminal and a second terminal respectively according to the type of the resource allocation request. In step S203A, feedback information about the allocation result is acquired from at least one of the first terminal and the second terminal. In step S204A, information about whether to modify the allocated first resource to a second resource is fed back to the first terminal according to the feedback information.

In some embodiments, the first terminal can be the patient side terminal, the second terminal can be the doctor side terminal, and the resource allocation request can be a request for seeing a doctor.

Embodiments of the present disclosure take considerations of feedback opinions on resource allocation from different terminals, which not only optimizes the resource allocation modes, but also satisfies the requirement of the users corresponding to the terminals, thereby reducing occurrence probability of disputes.

FIG. 2B is a schematic flowchart of a medical resource allocation method provided in some embodiments of the present disclosure. Herein, the first terminal is the patient side terminal, the second terminal is the doctor side terminal, and the resource allocation request is the request for seeing a doctor. By referring to FIG. 2B, it can be known that in step S201B, the request for seeing a doctor is received from the patient side terminal and a type of the request for seeing a doctor is determined. In step S202B, an allocation result of allocation of a designated-level hospital is fed back to the doctor side terminal and the patient side terminal respectively according to the type of the request for seeing a doctor. In step S203B, feedback information about the allocation result from at least one of the doctor side terminal and the patient side terminal is acquired. In step S204B, information about whether to refer the patient to a higher-level hospital is fed back to the patient side terminal according to the feedback information. For example, information about whether to refer the patient to a higher-level hospital can be fed back to the patient side terminal according to the feedback information from the doctor side terminal and/or the patient side terminal.

The embodiments of the present disclosure give considerations to diagnosis & treatment recommendations and opinions of both the doctor and the patient, so that the doctor has rights to select a patient, and make a diagnosis and give treatment to the patient or recommend him/her to a higher-level hospital, and the doctor has rights to decentralize the patients when being confronted with the pressure of being overcrowded. At the same time, the patients also obtain more benefits and freedoms. The patient has the right to decide whether to see a doctor in a current-level hospital. The embodiments of the present disclosure not only optimize the allocation way of the medical resources, but also respond to the wills of the patients, and satisfy the requirements of the patients as much as possible, so that it is helpful to improve the tense contradictory relationship between the doctors and the patients and reduce occurrence probability of disputes between the doctors and the patients.

The present disclosure will be described below in detail. Examples of embodiments of the present disclosure are shown in the figures, wherein same or similar reference marks always represent same or similar components, or components having same or similar functions. In addition, if detailed descriptions of the known technology are unnecessary for the features of the present disclosure, they would be omitted. Embodiments described by referring to the accompanying figures are for illustration, and are just used for explaining the present disclosure, but cannot be explained as a limitation to the present disclosure.

Those skilled in the art can understand that, unless otherwise defined, a variety of terms (including technical terms and scientific terms) used herein have the same meanings generally understood by those ordinary skilled in the art. It should be understood that those terms defined in a common dictionary should be understood as having meanings consistent with the meanings in the context of the prior art, and would not explained as idealized or over-formal meanings unless otherwise specially defined as here.

Those skilled in the art can understand that unless otherwise specified, the singular form of “a/an”, “one”, “the” used herein can also comprise the plural form. It shall be further understood that the word “comprise” or “include” used in the specification of the present disclosure means that the feature, integer, step, operation, element and/or component exist, but it does not exclude that one or more other features, integers, steps, operations, elements, components and/or groups thereof exist or can be added. It shall be understood that when it says that an element is “connected” or “coupled” to another element, it can be directly connected or coupled to other elements, or there may be an intermediate element. In addition, “connection” or “coupled” used herein can comprise wireless connection or wireless coupling. The word “and/or” used herein comprises all or any unit and all combinations of one or more listed associated items.

Technical solutions of the present disclosure and how the technical solutions of the present disclosure solve the above technical problem will be described in detail by using specific embodiments.

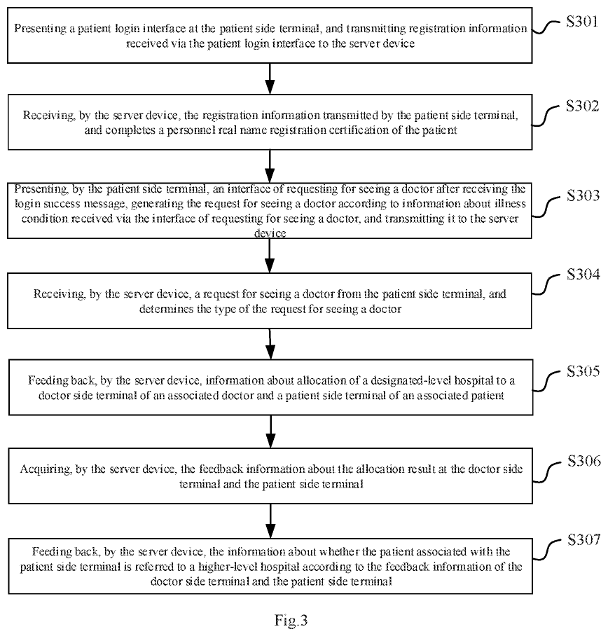

FIG. 3 is a schematic flowchart of an example of a medical resource allocation method provided in some embodiments of the present disclosure. This example provides a possible implementation for the medical resource allocation method of the embodiments of the present disclosure. As shown in FIG. 3, in step S301, a patient login interface is presented at the patient side terminal, and registration information is received via the patient login interface is transmitted to the server device.

In step S302, the server device receives login information transmitted by the patient side terminal, and completes a personnel real name registration certification of the patient.

In some embodiments, after receiving the login information transmitted by the patient side terminal, the server device analyzes personnel real name information of the patient from the login information. After that, the server device can compare the analyzed personnel real name information of the patient with the personnel real name information pre-registered by the patient. If a comparison result is that the analyzed personnel real name information and the pre-registered personnel real name information are consistent with each other, it is determined that the personnel real name registration certification of the patient is passed, so that a login success message is fed back to the patient side terminal. If the comparison result is that they are inconsistent, then it is determined that the personnel real name login certification of the patient fails, and thus a login failure message is fed back to the patient side terminal.

In step S303, an interface for requesting for seeing a doctor is presented after the patient side terminal receives the login success message. The request for seeing a doctor is generated according to information about illness condition received via the interface for requesting for seeing a doctor, and is transmitted to the server device.

In some embodiments, the interface for requesting for seeing a doctor is presented after the patient side terminal receives the login success message, and the information about illness condition of the patient is received via the interface for requesting for seeing a doctor. For example, the inventor(s) of the present disclosure considers that the illness of the patient is likely to become too severe to independently input the information about illness condition or the demand for seeing a doctor, the patient side terminal can receive the information about illness condition of the patient inputted by a person having a relationship with the patient (for example, a relative of the patient) via the interface for requesting for seeing a doctor.

The patient side terminal can generate a request for seeing a doctor according the information about illness condition of the patient, and transmits the request for seeing a doctor to the server device.

In step S304, the server device receives a request for seeing a doctor from the patient side terminal, and determines the type of the request for seeing a doctor.

The server device determines, according to the request for seeing a doctor, whether the request for seeing a doctor belongs to an emergency request for seeing a doctor or a normal request for seeing a doctor. If the request for seeing a doctor belongs to a normal request for seeing a doctor, the server device determines whether the request for seeing a doctor belongs to a normal preliminary request for seeing a doctor or belongs to a re-visiting request for seeing a doctor.

For example, the server device determines whether the patient needs an emergency treatment or rescue (i.e., treatment in emergency), according to the information about illness condition of the patient transmitted from the patient side terminal. If a determination result is “yes”, then it is determined that the request for seeing a doctor from the patient side terminal is the emergency request for seeing a doctor. Otherwise, it is determined that the request for seeing a doctor from the patient side terminal is the normal request for seeing a doctor (for example, outpatient request).

For example, if the server device determines from the information about the illness condition transmitted from the patient side terminal that traumatic bleeding occurs or the patient is having a high fever with a temperature exceeding X degrees (X is a preset threshold, for example, a real number higher than 39) and so on, then it is determined that the patient needs an emergency treatment or rescue, and thus it is determined that the request for seeing a doctor from the patient side terminal is the emergency request for seeing a doctor.

In some embodiments, with respect to the request for seeing a doctor which belongs to a normal request, the server device calls the information about illness condition of historical diagnosis of the patient relating to the request for seeing a doctor; and determines whether the information about illness condition in the request for seeing a doctor is consistent with information about illness condition of the historical diagnosis. If the information about illness condition in the request for seeing a doctor is consistent with information about illness condition of the historical diagnosis, it is determined that the request for seeing a doctor belongs to a normal re-visiting request for seeing a doctor. Otherwise, it is determined that the request for seeing a doctor belongs to a normal preliminary request for seeing a doctor.

In step S305, the server device feeds back information about allocation of a designated-level hospital to a doctor side terminal of an associated doctor and a patient side terminal of an associated patient.

Levels of the hospital are classified from low to high as a first-level hospital, a second-level hospital, a third-level hospital and a special-level hospital. For example, the community hospital is generally classified as the first-level hospital, the first-class hospital at grade 3 is classified as the special-level hospital.

If the request for seeing a doctor belongs to an emergency request, then information about a nearby hospital at any level is allocated, and allocation information is fed back to the doctor side terminal and the patient side terminal. If the request for seeing a doctor belongs to a normal preliminary request for seeing a doctor, then the first-level hospital is allocated, and the information about the first-level hospital is allocated to the doctor side terminal and the patient side terminal.

If the request for seeing a doctor belongs to a normal re-visiting request for seeing a doctor, then a corresponding-level hospital is allocated according to a degree of relevance between the information about illness condition associated with the patient at the patient side terminal and the stored information about illness condition of the historical diagnosis (of the patient), and information about allocation of a corresponding-level hospital is fed back.

In some embodiments, if the degree of relevance between the information about illness condition associated with the patient at the patient side terminal and the stored information about illness condition of the historical diagnosis (of the patient) is greater than or equal to a preset first threshold, then a hospital having the same level as the hospital of the previous diagnosis is allocated, and information about allocation of a hospital having the same level as the hospital of the previous treatment is fed back to the doctor side terminal and the patient side terminal.

If the degree of relevance between the information about illness condition associated with the patient at the patient side terminal and the stored information about illness condition of the historical diagnosis (of the patient) is smaller than the preset first threshold, then the first-level hospital is allocated, and information about allocation of the first-level hospital is fed back to the doctor side terminal and the patient side terminal.

In step S306, the server device acquires the feedback information about the allocation result from the doctor side terminal and the patient side terminal.

After the patient is allocated to the first-level hospital, the first-level hospital arranges a corresponding doctor to make a diagnosis and treatment for the patient. After the doctor in the first-level hospital makes a diagnosis for the patient, the doctor side terminal and the patient side terminal can generate feedback information about the allocation result based on one of the following three scenarios.

First, both the doctor and the patient agree the diagnosis result or solution of the doctor, and information about continuing the treatment or ending the treatment is transmitted to the server device via the doctor side terminal and the patient side terminal respectively.

Second, the doctor thinks that the patient's illness condition is severe and there is a potential of other possibilities of diseases, and the relevant information about the patient's diagnosis and test would be delivered to the present server device via the doctor side terminal. Then, the doctor sends recommendation to the server device according to the illness condition via the doctor side terminal that the patient should register at any level of hospital higher than the first-level hospital for registration and diagnosis.

Third, the patient does not accept the diagnosis result of the doctor in the first-level hospital, but the doctor thinks it is unnecessary to recommend this patient to a higher-level hospital to see a doctor. At this time, the patient can deliver a referral application to the server device via the patient side terminal.

In S307, the server device feeds back the information about whether the patient associated with the patient side terminal is referred to a higher-level hospital to the patient side terminal according to the feedback information of the doctor side terminal and the patient side terminal.

In case that the request for seeing a doctor from the patient side terminal belongs to a normal preliminary request for seeing a doctor, if the server device receives a referral recommendation from the doctor side terminal, the server device reviews the referral recommendation. If the server device approves the referral recommendation, the server terminal feeds back to the patient side terminal the information about allowing the patient to be referred to any level of hospital higher than a hospital mentioned in the referral recommendation.

If the server device receives a complaint from the patient side terminal about non-feedback of the referral recommendation of the doctor side terminal or a complaint about the review of the referral recommendation being rejected, then the server device reviews the complaint. If the review of the complaint is approved, the server device feeds back to the patient side terminal the information about allowing the patient to be referred to a higher-level hospital (for example, a second-level hospital). In some embodiments, reviewing the complaint by the server device can comprise bringing a coordinating agency of a third party to review the complaint. For example, the complaint can be reviewed according to comments of the coordinating agency of the third party.

If the referral request from the doctor side terminal and the complaint from the patient side terminal are not received, information about determining that the patient sees a doctor at the current-level hospital (for example, the first-level hospital) is fed back to the patient side terminal.

The above flows reflect the range of rights of corresponding doctors and the range of rights of corresponding patients upon being confronted with three different situations, i.e., a preliminary request for seeing a doctor, a re-visiting request for seeing a doctor, and an emergency request for seeing a doctor.

Based on a same inventive concept, there is provided in some embodiments of the present disclosure a resource allocation apparatus.

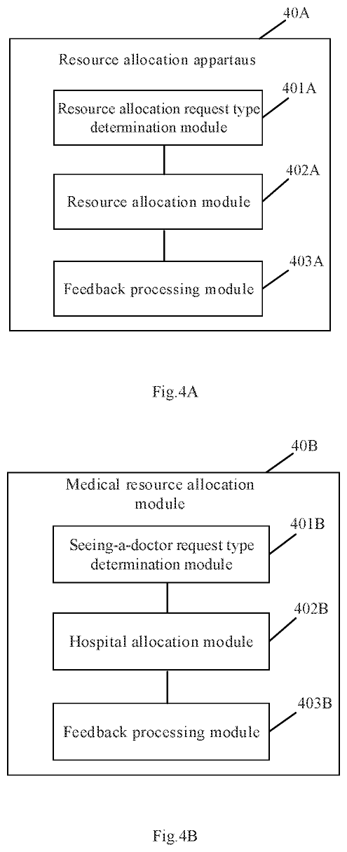

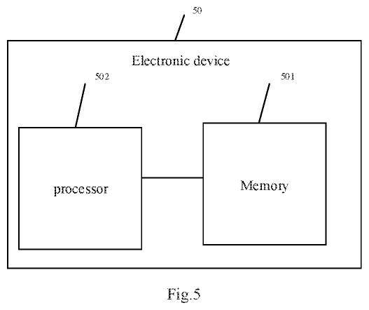

FIG. 4A is a schematic diagram of structure of a resource allocation apparatus provided in some embodiments of the present disclosure. The apparatus 40A comprises a resource allocation request type determination module 401A, a resource allocation module 402A and a feedback processing module 403A.

Herein, the resource allocation request type determination module 401A can be configured to receive a resource allocation request of a first terminal and determine a type of the resource allocation request.

The resource allocation module 402A can be configured to feed back an allocation result of an allocation of a first resource to the first terminal and a second terminal respectively.

The feedback processing module 403A can be configured to acquire feedback information about the allocation result from at least one of the first terminal and the second terminal, and feedback to the first terminal the information about whether the allocated first resource is modified in a second resource, according to the feedback information.

In some embodiments, the first terminal can be a patient side terminal, the second terminal can be a doctor side terminal, and the resource allocation request can be a request for seeing a doctor.

The embodiments of the present disclosure give considerations to feedback opinions on resource allocation from different terminals, which not only optimizes the allocation ways of resources, but also satisfies the requirement of the user corresponding to the terminal as much as possible, and reduces occurrence probability of disputes.

FIG. 4B is a schematic diagram of structure of a medical resource allocation apparatus provided in some embodiments of the present disclosure. The apparatus 40B comprises a seeing-a-doctor request type determination module 401B, a hospital allocation module 402B and a feedback processing module 403B.

Herein, the seeing-a-doctor request type determination module 401B can be configured to receive a request for seeing a doctor from the patient side terminal, and determine a type of the request for seeing a doctor.

The hospital allocation module 402B can be configured to feed back information about allocation of a designated-level hospital to the doctor side terminal and the patient side terminal respectively, according to the type of the request for seeing a doctor determined by the seeing-a-doctor request type determination module 401.

The feedback processing module 403B can be configured to acquire the feedback information about the allocation result (of the hospital allocation module 402B) at the doctor side terminal and the patient side terminal, and feedback to the patient side terminal the information about whether its associated patient is referred to an higher-level hospital, according to the doctor side terminal and the patient side terminal.

In some embodiments, the type of the request for seeing a doctor can be classified as a preliminary request for seeing a doctor, a re-visiting request for seeing a doctor, or a normal request for seeing a doctor and an emergency request for seeing a doctor. For example, the type of the request for seeing a doctor can be sub-classified as types such as a normal preliminary request for seeing a doctor, a normal re-visiting request for seeing a doctor, a preliminary emergency request for seeing a doctor or an emergency re-visiting request for seeing a doctor and so on.

In some embodiments, the hospital allocation module 402B can be configured to allocate information about hospitals of any level nearby if the request for seeing a doctor belongs to an emergency request, and feed back information about allocation of the first-level hospital to the doctor side terminal and the patient side terminal if the request for seeing a doctor belongs to a normal preliminary request. In case that the request for seeing a doctor belongs to a normal re-visiting request for seeing a doctor, if a degree of relevance of the information about illness condition of the patient side terminal and the stored information about illness condition of historical diagnosis is greater than or equal to a preset first threshold, information about allocation of a hospital having a same level as the hospital of the previous diagnosis is fed back to the doctor side terminal and the patient side terminal. If the degree of relevance of the information about illness condition of the patient side terminal and the stored information about illness condition of historical diagnosis is smaller than the preset first threshold, the information about allocation of the first-level hospital is fed back to the doctor side terminal and the patient side terminal.

In some embodiments, in case that the request for seeing a doctor from the patient side terminal belongs to a normal preliminary request for seeing a doctor, the feedback processing module 403B can be configured to review the referral recommendation if a referral recommendation of the doctor side terminal is received, and feed back to the patient side terminal the information about allowing the patient to be referred to any level of hospital higher than a hospital mentioned in the referral recommendation when the review of the referral recommendation is approved.

In some embodiment, the feedback processing module 403B generates one of the following two results through smart filtering or manual analyzing process or combination of the smart filtering and the manual analyzing process. The first one of the results is approving a referral application submitted by a doctor in the first-level hospital through the doctor side terminal. The patient side can register at a corresponding level hospital. The second one of the results is rejecting the referral application submitted by the doctor side terminal. At this time, the patient has a right to raise an objection to the rejecting result through the patient side terminal, i.e., complaining to the feedback processing module 403.

If the feedback processing module 403B receives the complaint of the patient side terminal about non-feedback of a referral recommendation from the doctor side terminal or the complaint about rejection of the review of the referral recommendation, the feedback processing module 403B reviews the complaint. If the review of the complaint is approved, the feedback processing module 403B feeds back to the patient side terminal the information about allowing the patient being referred to the second-level hospital.

It needs to be noted that if the doctor considers there is no necessary to recommend the patient to a higher-level hospital to see a doctor or the submitted referral application is rejected, the patient can still propose a complaint to the feedback processing module 403B voluntarily. If the feedback processing module 403B accepts the complaint of the patient, the patient can visit a higher-level hospital to see a doctor. Thus, it can be seen that the medical resource allocation apparatus 40B provided in the embodiments of the present disclosure gives a full right to the patient to select a level of a hospital for seeing a doctor.

In some embodiments, the feedback processing module 403B herein can be disposed in a coordinating agency of a third party, which would estimate, decide or review specific information received by the feedback processing module 403B, and generate feedback information.

The medial resource allocation apparatus 40B provided in the embodiments of the present disclosure is pre-stored with information about different levels of hospitals including the first-level hospital, the second-level hospital, the third-level hospital and the special-level hospital. The levels are classified as four types of levels. At the same time, the three modules of the seeing-a-doctor request type determination module 401B, the hospital allocation module 402B and the feedback processing module 403B are established, so as to effectively handle the problem when disagreement occurs between a personal requirement of a patient and a doctor diagnosis in the processes of the preliminary request for seeing a doctor and a re-visiting request for seeing a doctor. The patient's thoughts are taken into consideration in the feedback processing module in a user-friendly way, and the patient can complain and apply for a higher-level hospital to see a doctor in the case of not reaching a consensus with the doctor.

The embodiments of the present can decentralize the patients. Due to the existence of the user-friendly feedback processing module 403, more benefits and freedoms of the patients are guaranteed, which can reduce disputes between doctors and patients to a certain extent.

Based on a same inventive concept, there is provided an electronic device 50 in some embodiments of the present disclosure, comprising: a memory 501 and a processor 502 electrically connected with each other, and at least one program stored in the memory 501 and configured to implement the medial resource allocation method provided in the embodiments of the present disclosure when being executed by the processor 502.

Based on a same inventive concept, there is provided in some embodiments of the present disclosure a computer readable storage medium upon which a computer program is stored. The computer program implements the medical resource allocation method provided in the embodiments of the present disclosure when being executed by the processor.

By applying the embodiments of the present disclosure, following beneficial effects would be realized.

The present disclosure arranges a plurality of patients who are requesting for seeing a doctor at a same starting point. Both seeing a doctor and registering at a hospital need to start from a designated-level hospital (for example, seeing a doctor starts from a primary first-level hospital), which effectively integrates the medical resources of respective levels of hospitals.

The present disclosure gives considerations to diagnosis recommendations and opinions of both a doctor and a patient, so that the doctor has the right to decentralize the patients when being confronted with the pressure of being overcrowded. At the same time, the patient would obtain more benefits and freedoms, and the patient has the right to decide whether to see a doctor in the current-level hospital. The present disclosure not only optimizes the allocation ways of the medical resources, but also improves the tense contradictory relationship between the doctor and the patient, and reduces occurrence probability of disputes between the doctor and the patient.

Those skilled in the art can understand that, various operations, methods, and steps, measures and solutions in flows having already been discussed in the present disclosure can be altered, modified, combined or deleted. Further, various operations, methods, and steps, measures and solutions in flows having already been discussed in the present disclosure can also be altered, modified, rearranged, discomposed, combined or deleted. Further, the prior art having various operations, methods, and steps, measures and solutions in flows having already been discussed in the present disclosure can also be altered, modified, rearranged, discomposed, combined or deleted.

Terms of “first”, “second” are just for the purpose of description, but cannot be understood as indicating or suggesting relative importance or impliedly indicating the amount of technical features referred to. Thus, a feature defined by “first”, “second” can explicitly or impliedly comprise one or more features. In the description of the present disclosure, unless otherwise clarified, “a plurality of” means two or more than two.

It shall be understood that although respective steps in flow diagrams of the figures are shown in sequence according to as indicated by an arrow, these steps are not necessarily executed in sequence according to the order as indicated by an arrow. Unless other explicitly described in the text, execution of these steps is not limited to a strict sequence, and these steps can be executed in other sequences. Furthermore, at least a part of steps in the flow diagrams of the figures can comprise a plurality of sub-steps or a plurality of phases. These steps or phases are not necessarily executed and completed at the same time, but can be executed at different times. These steps or phases are not necessarily in sequence, but can be executed with other steps or at least a part of sub-steps or phases of other steps by turns or alternatively.

The above descriptions are a part of implementations of the present disclosure. It shall be pointed out that, for those ordinary skilled in the art, several improvement and modifications can further be made without departing from the principle of the present disclosure, and these improvements and modifications shall also be deemed as the protection scope of the present disclosure.

What is claimed is:

1. A resource allocation method, comprising:
receiving a resource allocation request of a first terminal and determining a type of the resource allocation request;
feeding back, according to the type of the resource allocation request, an allocation result of an allocation of a first resource to the first terminal and a second terminal respectively;
acquiring feedback information about the allocation result from at least one of the first terminal and the second terminal;
feeding back to the first terminal information about whether to modify the allocated first resource to a second resource according to the feedback information.

2. The resource allocation method according to claim 1, wherein the first terminal is a patient side terminal, the second terminal is a doctor side terminal, and the resource allocation request is a request for seeing a doctor, and the resource allocation method further comprises:
receiving the request for seeing a doctor from the patient side terminal and determining a type of the request for seeing a doctor;
feeding back the allocation result of allocation of a designated-level hospital to the doctor side terminal and the patient side terminal respectively, according to the type of the request for seeing a doctor;
acquiring feedback information about the allocation result from at least one of the doctor side terminal and the patient side terminal;
feeding back to the patient side terminal information about whether to refer the patient to a higher-level hospital, according to the feedback information.

3. The resource allocation method according to claim 2, wherein receiving the request for seeing a doctor from the patient side terminal and determining a type of the request for seeing a doctor comprises:
determining the type of the request for seeing a doctor as an emergency request for seeing a doctor, a normal preliminary request for seeing a doctor or a normal re-visiting request for seeing a doctor.

4. The resource allocation method according to claim 2, wherein feeding back the allocation result of allocation of a designated-level hospital to the doctor side terminal and the patient side terminal respectively according to the type of the request for seeing a doctor comprises:
feeding back the allocation result of allocation of a hospital nearby if the type of the request for seeing a doctor is the emergency request for seeing a doctor;
feeding back the allocation result of allocation of a first-level hospital to the doctor side terminal and the patient side terminal if the request for seeing a doctor is the normal preliminary request for seeing a doctor;
feeding back the allocation result of the allocated hospital to the doctor side terminal and the patient side terminal according to a degree of relevance between information about illness condition of a patient associated with the patient side terminal and stored information about illness condition of historical diagnosis, if the request for seeing a doctor is a normal re-visiting request for seeing a doctor.

5. The resource allocation method according to claim 4, wherein the feeding back the allocation result of the allocated hospital to the doctor side terminal and the patient side terminal according to a degree of relevance between information about illness condition of a patient associated with the patient side terminal and stored information about illness condition of historical diagnosis comprises:
feeding back information about allocation of a hospital having a same level as a hospital of a previous diagnosis, if the degree of relevance between information about illness condition at the patient side terminal and the stored information about illness condition of historical diagnosis is greater than or equal to a preset first threshold;
feeding back information about allocation of a first-level hospital to the doctor side terminal and the patient side terminal, if the degree of relevance between information about illness condition at the patient side terminal and the stored information about illness condition of historical diagnosis is smaller than the preset first threshold.

6. The resource allocation method according to claim 4, wherein feeding back to the patient side terminal information about whether to refer the patient to a higher-level hospital, according to the feedback information comprises:
in case that the request for seeing a doctor belongs to a normal preliminary request for seeing a doctor, if a referral recommendation from the doctor side terminal is received, reviewing the referral recommendation; and if the review of the referral recommendation is approved, feeding back to the patient side terminal information about allowing the patient to be referred to any level of hospital higher than a hospital mentioned in the referral recommendation;
in case that a complaint from the patient side terminal about non-feedback of the referral recommendation from the doctor side terminal or a complaint about the review of the referral recommendation being rejected, reviewing the complaint; if the review of the complaint is approved, feeding back to the patient side terminal the information about allowing the patient to be referred to a higher-level hospital;
in case that the referral request of the doctor side terminal and the complaint of the patient side terminal are not received, feeding back to the patient side terminal information about determining that the patient sees a doctor in a first-level hospital.

7. The resource allocation method according to claim 6, wherein reviewing the complaint comprises:
reviewing the complaint according to opinions of a coordinating agency of a third party.

8. A resource allocation apparatus, comprising:
a resource allocation request type determination module, configured to receive a resource allocation request of a first terminal and determine a type of the resource allocation request;
a resource allocation module, configured to feed back an allocation result of an allocation of a first resource to the first terminal and a second terminal respectively, according to the type of the resource allocation request;
a feedback processing module, configured to acquire feedback information about the allocation result from at least one of the first terminal and the second terminal, and feed back to the first terminal information about whether to modify the allocated first resource to a second resource according to the feedback information.

9. The resource allocation apparatus according to claim 8, wherein the first terminal is a patient side terminal, the second terminal is a doctor side terminal, and the resource allocation request is a request for seeing a doctor, and the resource allocation apparatus comprises:
a seeing-a-doctor request type determination module, configured to receive the request for seeing a doctor from the patient side terminal and determine the type of the request for seeing a doctor;
a hospital allocation module, configured to feed back the allocation result of an allocation of a designated-level hospital to the doctor side terminal and the patient side terminal respectively, according to the type of the request for seeing a doctor;
a feedback processing module, configured to acquire feedback information about the allocation result from at least one of the doctor side terminal and the patient side terminal, and feed back to the patient side terminal information about whether to refer the patient to a higher-level hospital, according to the feedback information.

10. The resource allocation apparatus according to claim 9, wherein the seeing-a-doctor request type determination module is further configured to:
determine the type of the request for seeing a doctor as an emergency request for seeing a doctor, a normal preliminary request for seeing a doctor or a normal re-visiting request for seeing a doctor.

11. The resource allocation apparatus according to claim 9, wherein the hospital allocation module is further configured to:
feed back information about allocation of a hospital nearby if the type of the request for seeing a doctor is the emergency request for seeing a doctor;
feed back information about allocation of a first-level hospital to the doctor side terminal and the patient side terminal if the request for seeing a doctor is the normal preliminary request for seeing a doctor;
feed back information about an allocated hospital to the doctor side terminal and the patient side terminal according to a degree of relevance between information about illness condition of a patient associated with the patient side terminal and stored information about illness condition of historical diagnosis, if the request for seeing a doctor is the normal re-visiting request for seeing a doctor.

12. The resource allocation apparatus according to claim 11, wherein hospital allocation module is further configured to:
feed back information about allocation of a hospital having a same level as a hospital of a previous diagnosis, if the degree of relevance between information about illness condition at the patient side terminal and the stored information about illness condition of historical diagnosis is greater than or equal to a preset first threshold;
feed back information about allocation of a first-level hospital to the doctor side terminal and the patient side terminal, if the degree of relevance between information about illness condition at the patient side terminal and the stored information about illness condition of historical diagnosis is smaller than the preset first threshold.

13. The resource allocation apparatus according to claim 11, wherein the feedback processing module is further configured to:
in case that the request for seeing a doctor belongs to the normal preliminary request for seeing a doctor, if a referral recommendation from the doctor side terminal is received, review the referral recommendation; and if the review of the referral recommendation is approved, feed back to the patient side terminal information about allowing the patient to be referred to any level of hospital higher than a hospital mentioned in the referral recommendation;
in case that a complaint from the patient side terminal about non-feedback of the referral recommendation from the doctor side terminal or a complaint about the review of the referral recommendation being rejected, review the complaint; if the review of the complaint is approved, feed back to the patient side terminal the information about allowing the patient to be referred to a higher-level hospital;
in case that the referral request of the doctor side terminal and the complaint of the patient side terminal are not received, feed back to the patient side terminal information about determining that the patient sees a doctor in a first-level hospital.

14. The resource allocation apparatus according to claim 13, wherein reviewing the complaint comprises:
reviewing the complaint according to opinions of a coordinating agency of a third party.

15. An electronic device, comprising:
a memory and a processor electrically connected with each other;
at least one program, stored in the memory and configured to implement the resource allocation method according to claim 1 when being executed by the processor.

16. The electrode device according to claim 15, wherein the electronic device is disposed in a coordinating agency of a third party.

17. A resource allocation system, comprising: a first terminal, a second terminal and a server device, wherein the server device of the resource allocation system has a communication connection with the first terminal and the second terminal respectively; and the server device comprises the resource allocation apparatus according to claim 8.

18. A computer readable storage medium, upon which a computer program is stored, wherein the computer program implements the resource allocation method according to claim 1 when being executed by a processor.
